(19) United States
(12) Patent Application Publication
(10) Pub. No.: US 2025/0116581 A1
(43) Pub. Date: Apr. 10, 2025

(54) SAMPLING EQUIPMENT AND SAMPLING METHOD

(71) Applicant: SHANGHAI ZKW MOLECULAR BREEDING TECHNOLOGY CO., LTD., Shanghai (CN)

(72) Inventors: Yu FANG, Shanghai (CN); Pan WANG, Shanghai (CN); Wei ZHOU, Shanghai (CN)

(73) Assignee: SHANGHAI ZKW MOLECULAR BREEDING TECHNOLOGY CO., LTD., Shanghai (CN)

(21) Appl. No.: 18/836,206

(22) PCT Filed: Jun. 7, 2023
(86) PCT No.: PCT/CN2023/098879

§ 371 Date: Aug. 6, 2024

(30) Foreign Application Priority Data
Nov. 1, 2022 (CN) 202211358278.7

(51) Int. Cl. G01N 1/28; G01N 1/08; G01N 33/00

(52) U.S. Cl. CPC G01N 1/286; G01N 1/08; G01N 33/0098; G01N 2001/2866; G01N 2001/2873

(57) ABSTRACT

An embodiment of the present disclosure provides a sampling equipment and a sampling method, which relates to the field of plant sampling technology, and the sampling equipment includes a sampling tool, a first driving assembly, and a crushing component. The sampling tool is configured to separate a sample from an object to be sampled, is formed with a crushing portion, and is disposed at the first driving assembly. The first driving assembly is configured to drive the sampling tool to move relative to the crushing component to crush the sample. The sampling equipment in the embodiment of the present disclosure reduces the total time for sampling and crushing the plant. Therefore, it can better link up to an existing high-throughput automated DNA extraction equipment and improve the efficiency of plant-related molecular biology research.

### CROSS-REFERENCE TO RELATED APPLICATIONS

The present disclosure is based on Chinese patent application No. 202211358278.7, field on Nov. 1, 2022, and claims priority to the Chinese patent application, which is incorporated herein by reference in its entirety.

### TECHNICAL FIELD

The present disclosure relates to the field of plant sampling technology, and in particular, to a sampling equipment and a sampling method.

### BACKGROUND

By sampling a plant, the collected plant samples are further tested. In a related technology, the sampling and crushing of plant leaves cannot link up to an existing high-throughput DNA automatic extraction equipment, which may affect the efficiency of plant-related molecular biology research.

### SUMMARY

In view of the above, an embodiment of the present disclosure provides a sampling equipment and a sampling method to improve the efficiency of plant-related molecular biology research.

To achieve the above purpose, a first aspect of an embodiment of the present disclosure provides a sampling equipment, including:
a sampling tool configured to separate a sample from an object to be sampled, and formed with a crushing portion;
a first driving assembly, wherein the sampling tool is disposed at the first driving assembly;
a crushing component, wherein the first driving assembly configured to drive the sampling tool to move relative to the crushing component to crush the sample.

In one embodiment, the crushing component may be formed with a sampling channel, and the crushing portion moves into the sampling channel to crush the sample.

In one embodiment, the crushing component may include:
a sample stage for placing the object to be sampled, wherein the sampling channel penetrates through the sample platform; and
a collection container disposed below the sample stage to collect the sample, wherein a closed end of the sampling channel is located at the collection container.

In one embodiment, the crushing portion may include at least one cutting edge.

In one embodiment, the cutting edge may surround to form a plurality of sub-sample areas.

In one embodiment, the at least one cutting edge may include a first cutting edge with an annular shape, the at least one cutting edge may include a second cutting edge disposed at an inner side of the first cutting edge, and the first cutting edge and the second cutting edge may surround to form the plurality of sub-sample areas.

In one embodiment, the first driving assembly may include:
a loading mechanism, wherein the sampling tool is disposed at the loading mechanism;
a separation device disposed at the loading mechanism and configured to separate the sampling tool from the loading mechanism to sample the object to be sampled on the crushing component.

In one embodiment, the separation device may be a firing device, and the loading mechanism may include:
a case, wherein the crushing component and the firing device are disposed at the case;
a loading body disposed at the case, wherein the sampling tool is installed at the loading body, and the firing device is configured to drive the sampling tool to separate from the loading body.

A second aspect of an embodiment of the present disclosure provides a sampling method applied to a sampling equipment, wherein the sampling equipment includes a sampling tool, a first driving assembly, and a crushing component, the sampling tool is configured to separate a sample from an object to be sampled, is formed with a crushing portion, and is disposed at the first driving assembly, and the first driving assembly is configured to drive the sampling tool to move relative to the crushing component to crush the sample, and the sampling method may include:
placing the object to be sampled at the crushing component;
activating the first driving assembly, and moving, by the crushing portion of the sampling tool, to at least contact with the object to be sampled through the first driving assembly to crush the object to be sampled.

In one embodiment, a sampling channel may be formed in the crushing component, and the crushing portion moves into the sampling channel to crush the sample, the first driving assembly may include a loading mechanism and a separation device, the sampling tool may be installed at the loading mechanism, and the separation device is disposed at the loading mechanism, the separation device may be configured to separate the sampling tool from the loading mechanism, and the step of activating the first driving assembly and moving, by the crushing portion of the sampling tool, to at least contact with the object to be sampled through the first driving assembly to crush the object to be sampled may include:
activating the separation device, so that the sampling tool moved out of the loading mechanism moves into the sampling channel.

The sampling equipment according to embodiments of the present disclosure separates the sample from the object to be sampled, and during the process of the first driving assembly 2 driving the sampling tool 1 to move relative to the crushing component 3 to sample the object to be sampled, the relative movement between the crushing portion 11 and the crushing component 3 can crush the object to be sampled placed on the crushing component 3. By crushing the collected sample during the sampling process, it is no necessary to transfer the collected sample to other devices for crushing. Both sampling and crushing of the object to be sampled can be achieved by using original time for sampling the object to be sampled, thereby reducing the total time for sampling and crushing the object to be sampled. Exemplarily, the sampling equipment can be used to sample a plant, and by crushing the collected sample during the sampling process, it is no necessary to transfer the collected sample to other devices for crushing. The sampling and crushing of the plant can be achieved by using the original sampling time of the plant, thereby reducing the total time for sampling and crushing the plant. Therefore, it can better link up to the existing high-throughput automated DNA extraction equipment and improve the efficiency of plant-related molecular biology research.

### BRIEF DESCRIPTION OF DRAWINGS

FIG. 1 is a schematic structural diagram illustrating a sampling equipment according to an embodiment of the present disclosure, in which a structure within a case is not shown;

FIG. 2 is a schematic structural diagram illustrating a sampling equipment according to an embodiment of the present disclosure, in which a structure within a case is shown, and part of the case is not shown;

FIG. 3 is an enlarged view of position A in FIG. 2;

FIG. 4 is a schematic structural diagram illustrating a sampling tool according to an embodiment of the present disclosure;

FIG. 5 is a schematic diagram illustrating a cutting position of a sampling tool according to an embodiment of the present disclosure;

FIG. 6 is a cross-sectional view at position B-B in FIG. 5;

FIG. 7 is a layout diagram of a sampling channel according to an embodiment of the present disclosure.

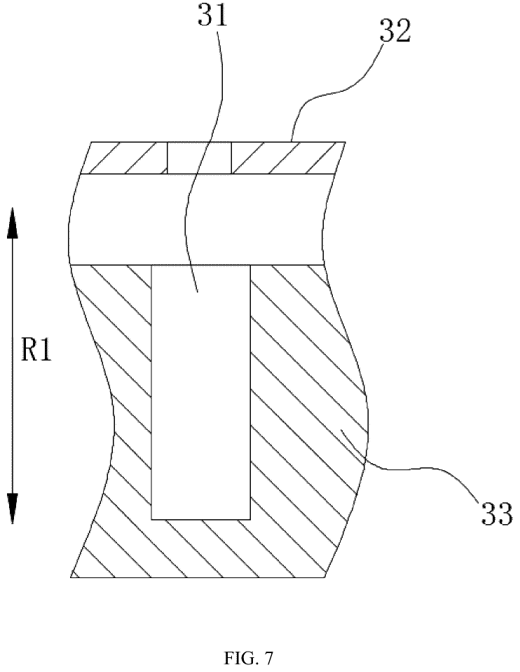

Explanation of the reference numerals:

sampling tool 1; crushing portion 11; sub-sample area 12; first cutting edge 13; second cutting edge 14; knife sleeve 15; crushing knife 16; first driving assembly 2; loading mechanism 21; case 211; loading body 212; separation device 22; crushing component 3; sampling channel 31; sample stage 32; collection container 33.

### DETAILED DESCRIPTION

It should be noted that, without conflict, the embodiments and the technical features in the embodiments can be combined with each other. The detailed description in a specific implementation should be understood as an explanation of the purpose of the present disclosure and should not be regarded as undue limitation on the present disclosure.

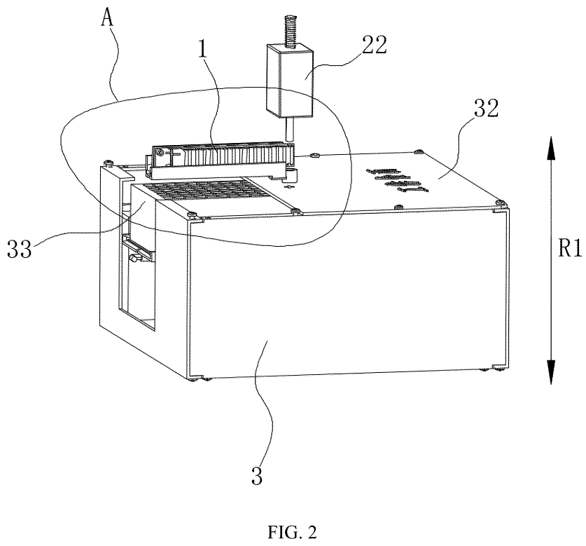

In the description of an embodiment of the present disclosure, “upper”, “lower”, “top”, “bottom”, orientation or position relationship is based on the orientation or position relationship shown in FIG. 2. It should be understood that these orientation terms are only for the convenience of describing the present disclosure and simplifying the description, but do not indicate or imply that the device or element referred to must have a specific orientation, be constructed and operated in a specific orientation, and therefore cannot be construed as a limitation of the present disclosure.

In an embodiment of the present disclosure, please refer to FIG. 2, in which an up-down direction refers to a direction indicated by an arrow R1.

As a part of the creative concept of the present disclosure, before describing the embodiments of the present disclosure, it is necessary to analyze the reasons why the sampling and crushing of plant leaves in the related technology cannot link up to the existing high-throughput DNA automatic extraction equipment. Through reasonable analysis, technical solution of embodiments of the present disclosure can be obtained.

In the related technology, after sampling the plant, collected samples need to be transferred to a corresponding crushing device for crushing, then a related test is performed. The sampling and crushing processes are performed separately, thereby making total time spent on sampling and crushing the plant long, low efficiency, and making it difficult to link up to the existing high-throughput automated DNA extraction equipment. Exemplarily, a hole punch used for sampling a leaf in the related technology can sample the leaf, but cannot crush it. The collected leaf samples need to be transferred to the corresponding crushing device for crushing, and then DNA extraction is performed.

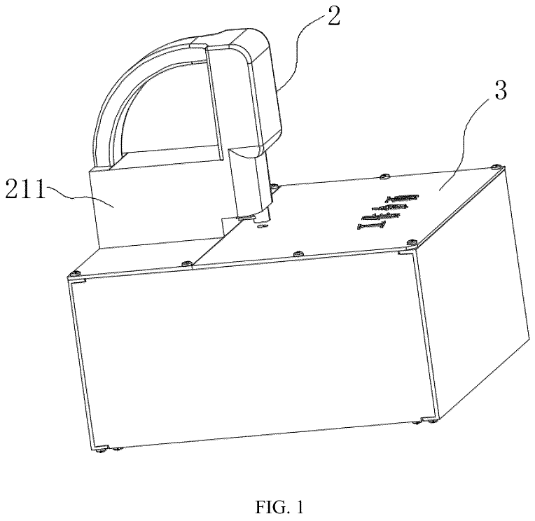

In view of this, an embodiment of the present disclosure provides a sampling equipment. Please refer to FIGS. 1 to 3. The sampling equipment includes a sampling tool 1, a first driving assembly 2, and a crushing component 3. The sampling tool 1 is configured to separate a sample from an object to be sampled, and is formed with a crushing portion 11. The sampling tool 1 is disposed at the first driving assembly 2. The first driving assembly 2 is configured to drive the sampling tool 1 to move relative to the crushing component 3, so that the crushing portion 11 crushes the sample. With such a structure, the sampling tool 1 separates the sample from the object to be sampled to achieve the sampling of the object to be sampled. In the process of the first driving assembly 2 driving the sampling tool 1 to move relative to the crushing component 3 to sample the object to be sampled, the relative movement between the crushing portion 11 and the crushing component 3 can crush the object to be sampled placed on the crushing component 3. By crushing the collected sample during the sampling process, it is no necessary to transfer the collected sample to other devices for crushing. The sampling and crushing of the object to be sampled can be achieved by using the original sampling time of the object to be sampled, thereby reducing the total time for sampling and crushing the object to be sampled. Exemplarily, the sampling equipment can be used to sample a plant, and by crushing the collected sample during the sampling process, it is no necessary to transfer the collected sample to other devices for crushing. The sampling and crushing of the plant can be achieved by using the original sampling time of the plant, thereby reducing the total time for sampling and crushing the plant. Therefore, it can better link up to the existing high-throughput automated DNA extraction equipment and improve the efficiency of plant-related molecular biology research.

In one embodiment, the sampling equipment can be used to sample leaves of a plant.

It should be noted that the sampling equipment can not only sample leaves, but also sample other roughly flaky parts of the plant. The object sampled by the sampling equipment is not limited to leaves of the plant.

It should be noted that sampling equipment is not limited to sample the plant.

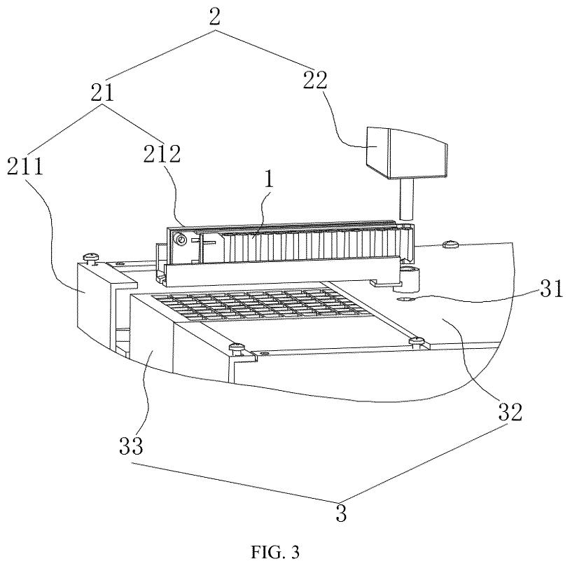

In one embodiment, please refer to FIG. 3, the crushing component 3 may be formed with a sampling channel 31, and the crushing portion 11 may move into the sampling channel 31 to crush the sample. With such a structure, the crushing portion 11 moves into the sampling channel 31 to crush a plant sample, and thus the crushed samples can be retained in the sampling channel 31 as much as possible to prevent the crushed samples from dispersing irregularly.

In one embodiment, the crushing component 3 may not be provided with the sampling channel 31, the plant may be placed on the crushing component 3, and a plane for carrying the plant on the crushing component 3 may cooperate with the crushing portion 11 to crush the plant on the crushing component 3.

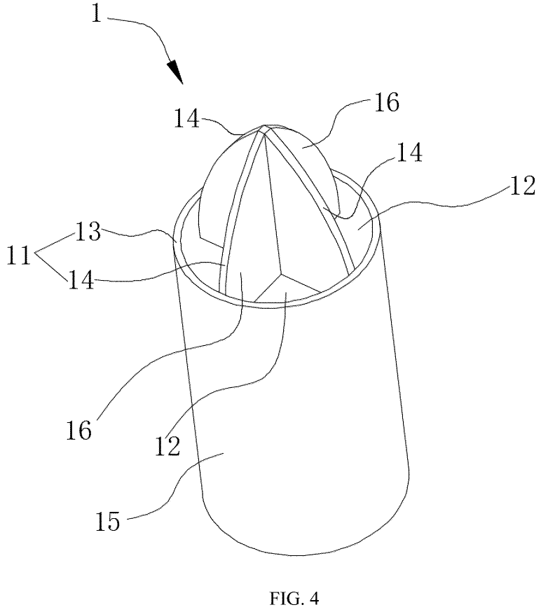

In one embodiment, please refer to FIGS. 4 to 6, the crushing portion 11 may include at least one cutting edge. With such a structure, the plant placed on the crushing component 3 can be cut up by the cutting edge.

In one embodiment, in a case that the crushing component 3 is not provided with the sampling channel 31, the plant on the crushing component 3 can be cut up by the cutting edge of the crushing portion 11.

In one embodiment, when the crushing portion 11 moves into the sampling channel 31, the plant sample in the sampling channel 31 may be cut up by the cutting edge and separated from the plant. The separated and cut-up plant may be brought into the sampling channel 31 by the crushing portion 11 for retaining.

In one embodiment, please refer to FIG. 3, the crushing component 3 may include a sample stage 32 and a collection container 33. The sample stage 32 may be used to place the object to be sampled, and the sampling channel 31 may penetrate through the sample stage 32. The collection container 33 may be disposed below the sample stage 32 to collect the sample, and a closed end of the sampling channel 31 is located at the collection container 33. With such a structure, in a case that the object to be sampled is a plant, the plant is placed on the sample stage 32, and the collection container 33 is located below the sample stage 32. The plant and the collection container 33 are isolated to a certain extent by the sample stage 32, and the collection container 33 collects the separated and crushed sample by the sampling channel, thereby preventing the collection container 33 from contacting unsampled parts of the plant on the sample stage 32 to a certain extent and reducing sample contamination. The sampling channel 31 penetrates through the sample stage 32, which facilitates the collected sample of the plant on the sample stage 32 to fall into the collection container 33 below through the sampling channel 31, thereby facilitating the collection of the separated and crushed sample. The closed end of the sampling channel 31 is located at the collection container 33, which means that the sampling channel 31 does not penetrate through the collection container 33, and thus the sample falling into the sampling channel can be better retained in the collection container 33, which is beneficial to sample collection.

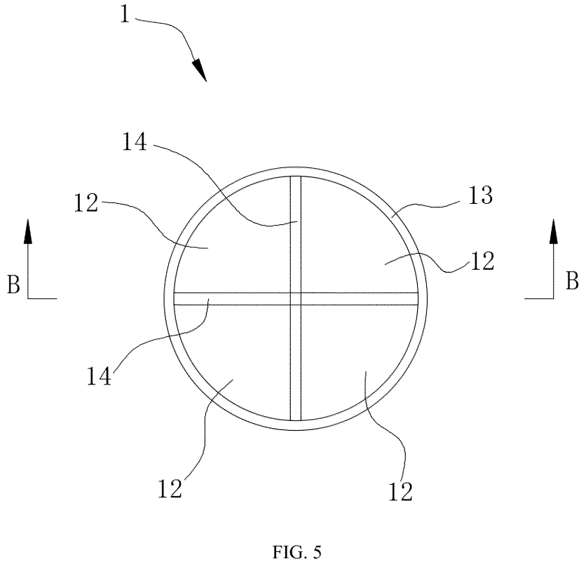

In one embodiment, please refer to FIGS. 4 and 5, the cutting edge may surround to form a plurality of sub-sample areas 12. With such a structure, since the cutting edge surrounds to form the plurality of sub-sample areas 12, a sample of a plant may be divided into a plurality of sub-samples corresponding to the sub-sample areas 12 by the cutting edge, thereby realizing the crushing of the sample.

It should be noted that “a plurality of” refers to two or more. Illustratively, it may be 2, 5, or 8.

In one embodiment, please refer to FIGS. 4 to 6, the at least one cutting edge may include a first cutting edge 13. The first cutting edge 13 may have an annular shape. With such a structure, the annular first cutting edge 13 may be beneficial to separating the sample from the plant.

In one embodiment, please refer to FIGS. 4 to 6, in which the at least one cutting edge may include a second cutting edge 14. The second cutting edge 14 may be disposed at an inner side of the first cutting edge 13. The first cutting edge 13 and the second cutting edge 14 may surround to form a plurality of sub-sample areas 12. With such a structure, the second cutting edge 14 is disposed at the inner side the first cutting edge 13, which means that the second cutting edge 14 is disposed in a space surrounded by the first cutting edge 13, the first cutting edge 13 and the second cutting edge 14 surround to form the plurality of sub-sample areas 12, and the sample is divided into a plurality of sub-samples corresponding to the sub-sample areas 12 by the cooperation of the first cutting edge 13 and the second cutting edge 14. The annular first cutting edge 13 may separate the sample from the plant.

It should be noted that the number of the first cutting edge 13 can be set according to actual needs. Exemplarily, the number of the first cutting edge 13 may be one. Exemplarily, there may be a plurality of the first cutting edges 13.

It should be noted that the number of second cutting edge 14 is not limited. Exemplarily, the number of the second cutting edge 14 may be 1, 2, 4 or 7.

Exemplarily, the number of the first cutting edge 13 and the second cutting edge 14 may be one, and the second cutting edge 14 may divide the space surrounded by the first cutting edge 13 into two sub-sample areas 12.

Exemplarily, please refer to FIGS. 4 to 6, the number of the first cutting edge 13 may be one, and the number of the second cutting edge 14 may be four. One first cutting edge 13 and four second cutting edges 14 may surround to form four sub-sample areas 12.

In one embodiment, please refer to FIGS. 4 to 6. the sampling tool 1 may include a knife sleeve 15 and a crushing knife 16 connected to an inner side of the knife sleeve 15. The first cutting edge 13 may be formed at the knife sleeve 15 and located at an end of the knife sleeve 15 facing the crushing knife 16. The second cutting edge 14 may be formed at the crushing knife 16. The sub-sample areas 12 surrounded by the first cutting edge 13 and the second cutting edge 14 may be connected to a space in the knife sleeve 15. With such a structure, when the sampling tool 1 remains in the sampling channel 31 along with the crushed samples, the sub-sample areas 12 may be connected to the space in the knife sleeve 15, which is convenient for an operator to extract the crushed sample in the sampling channel 31 from an end of the knife sleeve 15 away from the crushing knife 16 through the space within the knife sleeve 15 and the sub-sample areas 12.

In one embodiment, please refer to FIGS. 4 to 6, there are a plurality of the crushing knives 16. The plurality of crushing knives 16 may be disposed along a circumferential direction of the knife sleeve 15. All the crushing knives 16 are connected to each other along a radial direction of the knife sleeve 15 in a side away from the knife sleeve 15. In this way, the number of sub-sample areas 12 is equal to the number of crushing knives 16.

In one embodiment, please refer to FIGS. 4 to 6, the number of crushing knives 16 may be four.

In one embodiment, please refer to FIGS. 4 to 6, each crushing knife 16 may be formed with a second cutting edge 14.

In one embodiment, please refer to FIGS. 4 to 6, the second cutting edge 14 may be disposed at a side of the crushing knife 16 facing the knife sleeve 15 along the radial direction of the knife sleeve 15.

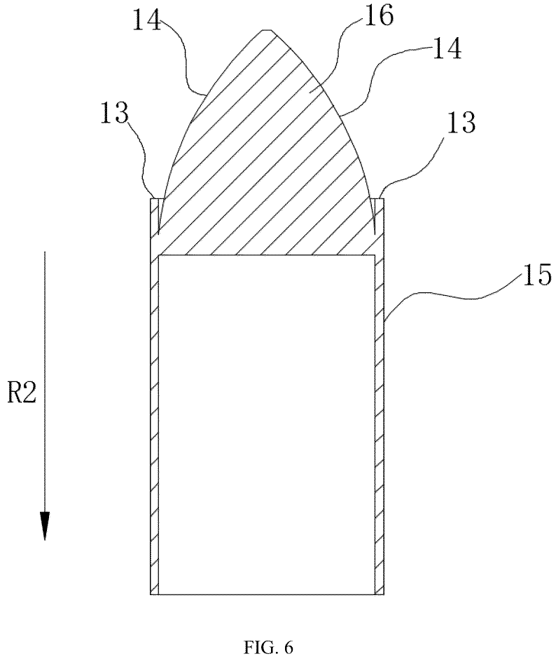

In one embodiment, please refer to FIG. 6, a size of the second cutting edge along the radial direction of the knife sleeve 15 may be a preset size, and a direction in which the crushing knife 16 points to the knife sleeve 15 along the axial direction of the knife sleeve 15 is a preset direction, and the preset size gradually increases in the preset direction. With such a structure, the second cutting edge 14 on the crushing knife 16 converges into a knife nose at an end of the crushing knife 16 that deviates from the preset direction. During a sampling process, the knife nose of the crushing knife 16 that deviates from the preset direction may penetrate the sample and the second cutting edge may gradually divide the sample, and the divided samples may be separated from the plant under an action of a first cutting edge.

In one embodiment, the preset direction may be the direction indicated by arrow R2 in FIG. 6.

In one embodiment, please refer to FIG. 6, the first cutting edge and the second cutting edge may be spaced apart. With such a structure, the first cutting edge may be arranged in a complete annular shape, which facilitates the separation of the sample from the plant through the first cutting edge.

In one embodiment, the crushing portion 11 does not necessarily have a cutting edge structure. A cutting edge may be disposed at an opening of the sampling channel 31 of the crushing component 3. The first driving assembly 2 may drive a plurality of sampling knives 1 to move into the sampling channel 31 through corresponding sub-sample areas, thereby realizing the cutting and crushing of the sample.

In one embodiment, the collection container 33 may be a 96 orifice plate or a 48 orifice plate.

In one embodiment, the collection container 33 may be a polymerase chain reaction (PCR) plate. A bottom of the PCR plate may have cone shape.

In one embodiment, the sampling equipment may further include a second driving assembly. The second driving assembly may be configured to drive the collection container 33 to move, so that different spaces in the collection container 33 may be served as parts of the sampling channel 31 to receive samples.

In one embodiment, please refer to FIG. 3, the first driving assembly 2 may include a loading mechanism 21 and a separation device 22. The sampling tool 1 may be installed at the loading mechanism 21, and the separation device 22 may be disposed at the loading mechanism 21. The separation device 22 may be configured to separate the sampling tool 1 from the loading mechanism 21 to sample an object to be sampled on the crushing component 3. With such a structure, the sampling can be performed by separating the sampling tool 1 from the loading mechanism 21 through the separation device 22. Since the sampling tool 1 is separated from the loading mechanism 21 during the sampling process, the loading mechanism 21 will load other sampling tools 1 to a position where the sampling can be performed, so as to perform a next sampling. Thus, the same sampling tool 1 may be not used for different samplings, thereby avoiding mutual contamination of samples collected in a plurality of times due to the same sampling tool 1.

In one embodiment, the sampling tool 1 can be drivingly connected to the first driving assembly 2, and the sampling tool 1 can be driven by the first driving assembly 2 to perform repeated samplings.

In one embodiment, please refer to FIG. 3, the separation device 22 may be a firing device.

In one embodiment, please refer to FIG. 3, the loading mechanism 21 may include a case 211 and a loading body 212. The crushing component 3 and the firing device may be disposed at the case 211. The loading body 212 may be disposed at the case 211, the sampling tool 1 may be installed at the loading body 212, and the firing device may be configured to drive the sampling tool 1 to separate from the loading body 212. With such a structure, the loading body 212, the crushing component 3, and the firing device can be disposed at the case 211, so that the loading body 212, the crushing component 3, and the firing device can be supported by the case 211, and the firing device disposed on the case 211 may be configured to bombard the sampling tool 1 in the loading body 212 to separate the sampling tool 1 from the loading body 212, and the plant on the crushing component 3 may be sampled through the separated sampling tool 1.

In one embodiment, the loading body 212 may include a body and a clamping member rotationally connected to the body. The body may be installed at the case 211. The sampling tool 1 may be installed at the body. The separation device 22 may be installed at the body or the case 211. The separation device 22 may drive the clamping member to move to clamp or release the sampling tool 1. When the separation device 22 releases, the sampling tool 1 falls downward under the action of gravity, thereby sampling the plant below the sampling tool 1.

An embodiment of the present disclosure provides a sampling method, which is applied to the sampling equipment. The sampling method includes placing an object to be sampled at the crushing component 3; and activating the first driving assembly 2, and moving, by the crushing portion 11 of the sampling tool 1 to at least contact with the object to be sampled through the first driving assembly 2 to crush the object to be sampled.

The sampling tool 1 may move relative to the crushing component 3 to sample the plant on the crushing component 3 by activating the first driving assembly 2.

In one embodiment, the step of activating the first driving assembly 2, and moving, by the crushing portion 11 of the sampling tool 1, to at least contact with the object to be sampled through the first driving assembly 2 to crush the object to be sampled may include activating the separation device 22, so that the sampling tool 1 moved out of the loading mechanism 21 moves into the sampling channel 31.

By activating the separation device 22, the sampling tool 1 can be separated from the loading mechanism 21 to avoid mutual contamination of samples obtained from a plurality of samplings due to the same sampling knife.

In one embodiment, the case 211 may be formed with a handle, and the handle may be disposed at an outer side of the case 211, thereby facilitating carrying the sampling equipment through the handle.

In one embodiment, the case 211 may be formed with an opening, and the opening may be aligned with the loading body 212 along an axial direction of the opening. In this way, it is convenient to disassemble and install the loading body 212 to load the sampling tool 1 into the loading body 212.

The embodiments above are only preferred embodiments of the present disclosure and are not intended to limit the present disclosure. For those skilled in the art, the present disclosure may have various modifications and changes. Any modifications, equivalent replacements, improvements, etc. made within the spirit and principles of the present disclosure shall be included in the protection scope of the present disclosure.

1. A sampling equipment, comprising:
a sampling tool configured to separate a sample from an object to be sampled, and formed with a crushing portion;
a first driving assembly, wherein the sampling tool is disposed at the first driving assembly; and
a crushing component, wherein the first driving assembly configured to drive the sampling tool to move relative to the crushing component to crush the sample.

2. The sampling equipment of claim 1, wherein the crushing component is formed with a sampling channel, and the crushing portion moves into the sampling channel to crush the sample.

3. The sampling equipment of claim 2, wherein the crushing component comprises:
a sample stage for placing the object to be sampled, wherein the sampling channel penetrates through the sample platform; and
a collection container disposed below the sample stage to collect the sample, wherein a closed end of the sampling channel is located at the collection container.

4. The sampling equipment of claim 1, wherein the crushing portion comprises at least one cutting edge.

5. The sampling equipment of claim 4, wherein the cutting edge surrounds to form a plurality of sub-sample areas.

6. The sampling equipment of claim 5, wherein
the at least one cutting edge comprises a first cutting edge with an annular shape,
the at least one cutting edge comprises a second cutting edge disposed at an inner side of the first cutting edge, and
the first cutting edge and the second cutting edge surround to form the plurality of sub-sample areas.

7. The sampling equipment of claim 1, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

8. The sampling equipment of claim 7, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.

9. A sampling method, applied to a sampling equipment, wherein the sampling equipment comprises a sampling tool, a first driving assembly, and a crushing component, the sampling tool is configured to separate a sample from an object to be sampled, is formed with a crushing portion, and is disposed at the first driving assembly, and the first driving assembly is configured to drive the sampling tool to move relative to the crushing component to crush the sample, and the sampling method comprises:
placing the object to be sampled at the crushing component, and
activating the first driving assembly, and moving, by the crushing portion of the sampling tool, to at least contact with the object to be sampled through the first driving assembly to crush the object to be sampled.

10. The sampling method of claim 9, wherein
a sampling channel is formed in the crushing component, and the crushing portion moves into the sampling channel to crush the sample,
the first driving assembly comprises a loading mechanism and a separation device,
the sampling tool is installed at the loading mechanism, and the separation device is disposed at the loading mechanism,
the separation device is configured to separate the sampling tool from the loading mechanism, and
the step of activating the first driving assembly and moving, by the crushing portion of the sampling tool, to at least contact with the object to be sampled through the first driving assembly to crush the object to be sampled comprises:
activating the separation device, so that the sampling tool moved out of the loading mechanism moves into the sampling channel.

11. The sampling equipment of claim 2, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

12. The sampling equipment of claim 11, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.

13. The sampling equipment of claim 3, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

14. The sampling equipment of claim 13, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.

15. The sampling equipment of claim 4, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

16. The sampling equipment of claim 15, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.

17. The sampling equipment of claim 5, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

18. The sampling equipment of claim 17, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.

19. The sampling equipment of claim 6, wherein the first driving assembly comprises:
a loading mechanism, wherein the sampling tool is disposed at the loading mechanism; and
a separation device disposed at the loading mechanism and configured to separate the sampling tool from the loading mechanism to sample the object to be sampled on the crushing component.

20. The sampling equipment of claim 19, wherein the separation device is a firing device, and
wherein the loading mechanism comprises:
a case, wherein the crushing component and the firing device are disposed at the case; and
a loading body disposed at the case, wherein the sampling tool is installed at the loading body, and the firing device is configured to drive the sampling tool to separate from the loading body.
